(19) United States
(12) Patent Application Publication
(10) Pub. No.: US 2014/0275089 A1
(43) Pub. Date: Sep. 18, 2014

(54) 2-Carboxamide Cycloamino Urea Derivatives in Combination with HSP90 Inhibitors for the Treatment of Proliferative Diseases

(71) Applicant: NOVARTIS AG, Basel (CH)

(72) Inventors: Xizhong Huang, Southborogh, MA (US); Cornelia Quadt, Allschwil (CH); Hui-Qin Wang, Lexington, MA (US); Christine Fritsch, Steinbach (FR); Christian René Schnell, Hegenhein (FR)

(21) Appl. No.: 14/350,577

(22) PCT Filed: Oct. 11, 2012
(86) PCT No.: PCT/EP2012/070171

§ 371 Date: Apr. 9, 2014

Related U.S. Application Data
(60) Provisional application No. 61/547,308, filed on Oct. 14, 2011.

(51) Int. Cl. A61K 31/5377; A61K 31/4439

(52) U.S. Cl. CPC A61K 31/5377; A61K 31/4439
USPC 514/236.8; 514/342

(57) ABSTRACT

The present invention relates to a pharmaceutical combination comprising a 2-carboxamide cycloamino urea derivative compound of formula (I) and inhibitors of Heat Shock Protein 90, and the uses of such combinations in the treatment of proliferative diseases, more specifically PI3K dependent diseases, more specifically PI3K-alpha dependent diseases.

### FIELD OF THE INVENTION

The present invention relates to a pharmaceutical combination comprising a 2-carboxamide cycloamino urea derivative compound of formula (I) and inhibitors of Heat Shock Protein 90, and the uses of such combinations in the treatment of proliferative diseases, more specifically PI3K dependent diseases, more specifically PI3K-alpha dependent diseases.

### BACKGROUND OF THE INVENTION

The PI3K/Akt/mTOR pathway is an important, tightly regulated survival pathway for the normal cell. Phophatidylinositol 3-kinases (PI3Ks) are widely expressed lipid kinases that catalyze the transfer of phosphate to the D-3′ position of inositol lipids to produce phosphoinositol-3-phosphate (PIP), phosphoinositol-3,4-diphosphate (PIP₂) and phosphoinositol-3,4,5-triphosphate (PIP₃). These products of the PI3K-catalyzed reactions act as second messengers and have central roles in key cellular processes, including cell growth, differentiation, mobility, proliferation and survival.

Of the two Class 1 PI3Ks, Class 1A PI3Ks are heterodimers composed of a catalytic p110 subunit (α, β, δ isoforms) constitutively associated with a regulatory subunit that can be p85α, p55α, p50α, p85β or p55γ. The Class 1B sub-class has one family member, a heterodimer composed of a catalytic p110γ subunit associated with one of two regulatory subunits, p101 or p84 (Fruman et al., Annu Rev. Biochem. 67:481 (1998); Suire et al., Curr. Biol. 15:566 (2005)).

In many cases, PIP2 and PIP3 recruit AKT to the plasma membrane where it acts as a nodal point for many intracellular signaling pathways important for growth and survival (Fantl et al., Cell 69:413-423 (1992); Bader et al., Nature Rev. Cancer 5:921 (2005); Vivanco and Sawyer, Nature Rev. Cancer 2:489 (2002)). Aberrant regulation of PI3K, which often increases survival through AKT activation, is one of the most prevalent events in human cancer and has been shown to occur at multiple levels. The tumor suppressor gene PTEN, which dephosphorylates phosphoinositides at the 3′ position of the inositol ring and in so doing antagonizes PI3K activity, is functionally deleted in a variety of tumors. In other tumors, the genes for the p110α isoform, PIK3CA, and for AKT are amplified and increased protein expression of their gene products has been demonstrated in several human cancers. Further, somatic missense mutations in PIK3CA that activate downstream signaling pathways have been described at significant frequencies in a wide diversity of human cancers (Kang at el., Proc. Natl. Acad. Sci. USA 102:802 (2005); Samuels et al., Science 304:554 (2004); Samuels et al., Cancer Cell 7:561-573 (2005)). Thus, inhibitors of PI3K alpha are known to be of particular value in the treatment of proliferative disease and other disorders.

Further, heat shock protein 90 (Hsp90) is recognized as an anti-cancer target. Hsp90 is a highly abundant and essential protein which functions as a molecular chaperone to ensure the conformational stability, shape and function of client proteins. The Hsp90 family of chaperones is comprised of four members: Hsp90α and Hsp90β both located in the cytosol, GRP94 in the endoplasmic reticulum, and TRAP1 in the mitochondria. Hsp90 is an abundant cellular chaperone constituting about 1%-2% of total protein.

Among the stress proteins, Hsp90 is unique because it is not required for the biogenesis of most polypeptides. Hsp90 forms complexes with oncogenic proteins, called “client proteins”, which are conformationally labile signal transducers playing a critical role in growth control, cell survival and tissue development. Such binding prevents the degradation of these client proteins. A subset of Hsp90 client proteins, such as Raf, AKT, phospho-AKT, CDK4 and the EGFR family including ErbB2, are oncogenic signaling molecules critically involved in cell growth, differentiation and apoptosis, which are all processes important in cancer cells. Inhibition of the intrinsic ATPase activity of Hsp90 disrupts the Hsp90-client protein interaction resulting in their degradation via the ubiquitin proteasome pathway.

Hsp90 chaperones, which possess a conserved ATP-binding site at their N-terminal domain belong to a small ATPase sub-family known as the DNA Gyrase, Hsp90, Histidine Kinase and MutL (GHKL) sub-family. The chaperoning (folding) activity of Hsp90 depends on its ATPase activity which is weak for the isolated enzyme. However, it has been shown that the ATPase activity of Hsp90 is enhanced upon its association with proteins known as co-chaperones. Therefore, in vivo, Hsp90 proteins work as subunits of large, dynamic protein complexes. Hsp90 is essential for eukaryotic cell survival and is overexpressed in many tumors.

In spite of numerous treatment options for proliferative disease patients, there remains a need for effective and safe therapeutic agents and a need for their preferential use in combination therapy. Surprisingly, it has been found that specific 2-carboxamide cycloamino urea derivative compounds of formula (I), which have been described in WO 2010/029082, provoke strong anti-proliferative activity and an in vivo antitumor response in combination with Hsp90 inhibitors. Co-treatment of cancer cells with an Hsp90 inhibitor and PI3K inhibitor, particularly a highly specific PI3K alpha inhibitor compound of formula (I), is particularly effective since it combines inhibition of proximal pathway components such as receptor tyrosine kinases (mainly targeted through Hsp90 inhibition) with another inhibitor (PI3K inhibitor) that is also acting close to the top of the signaling cascade. An additional benefit of Hsp90 inhibition may arise from its effect on other signaling components within the PI3K/Akt/mTOR pathway, as for example on AKT and pAKT, and its broad effects on many client proteins.

### SUMMARY OF THE INVENTION

The present invention relates to a pharmaceutical combination comprising (a) a compound of formula (I),

[chemical structure]

wherein
A represents a heteroaryl selected from the group consisting of:

[chemical structure]

R¹ represents one of the following substituents: (1) unsubstituted or substituted, preferably substituted C₁-C₇-alkyl, wherein said substituents are independently selected from one or more, preferably one to nine of the following moieties: deuterium, fluoro, or one to two of the following moieties C₃-C₅-cycloalkyl; (2) optionally substituted C₃-C₅-cycloalkyl wherein said substituents are independently selected from one or more, preferably one to four of the following moieties: deuterium, C₁-C₄-alkyl (preferably methyl), fluoro, cyano, aminocarbonyl; (3) optionally substituted phenyl wherein said substituents are independently selected from one or more, preferably one to two of the following moieties: deuterium, halo, cyano, C₁-C₇-alkyl, C₁-C₇-alkylamino, di(C₁-C₇-alkyl)amino, C₁-C₇-alkylaminocarbonyl, di(C₁-C₇-alkyl)aminocarbonyl, C₁-C₇-alkoxy; (4) optionally mono- or di-substituted amine; wherein said substituents are independently selected from the following moieties: deuterium, C₁-C₇-alkyl (which is unsubstituted or substituted by one or more substituents selected from the group of deuterium, fluoro, chloro, hydroxy), phenylsulfonyl (which is unsubstituted or substituted by one or more, preferably one, C₁-C₇-alkyl, C₁-C₇-alkoxy, di(C₁-C₇-alkyl)amino-C₁-C₇-alkoxy); (5) substituted sulfonyl; wherein said substituent is selected from the following moieties: C₁-C₇-alkyl (which is unsubstituted or substituted by one or more substituents selected from the group of deuterium, fluoro), pyrrolidino, (which is unsubstituted or substituted by one or more substituents selected from the group of deuterium, hydroxy, oxo; particularly one oxo); (6) fluoro, chloro;
R² represents hydrogen;
R³ represents (1) hydrogen, (2) fluoro, chloro, (3) optionally substituted methyl, wherein said substituents are independently selected from one or more, preferably one to three of the following moieties: deuterium, fluoro, chloro, dimethylamino;
with the exception of (S)-Pyrrolidine-1,2-dicarboxylic acid 2-amide 1-({5-[2-(tert-butyl)-pyrimidin-4-yl]-4-methyl-thiazol-2-yl}-amide),
   or a pharmaceutically acceptable salt thereof; and (b) at least one Hsp90 inhibitor or a pharmaceutically acceptable salt thereof. Such combination may be for simultaneous, separate or sequential use for the treatment of a proliferative disease.

In the preferred embodiment, the pharmaceutical combination of the present invention comprises a compound of formula (I) selected from (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (“Compound A”) or a pharmaceutically acceptable salt thereof.

The pharmaceutical combination of the present invention includes at least one compound targeting, decreasing or inhibiting the intrinsic ATPase activity of Hsp90 and/or degrading, targeting, decreasing or inhibiting the Hsp90 client proteins via the ubiquitin proteosome pathway. Such compounds will be referred to as “Heat shock protein 90 inhibitors” or “Hsp90 inhibitors. Examples of Hsp90 inhibitors suitable for use in the present invention include, but are not limited to, the geldanamycin derivative, Tanespimycin (17-allylamino-17-demethoxygeldanamycin)(also known as KOS-953 and 17-AAG); Radicicol; 6-Chloro-9-(4-methoxy-3,5-dimethylpyridin-2-ylmethyl)-9H-purin-2-amine methanesulfonate (also known as CNF2024); IPI504; SNX5422; 5-(2,4-Dihydroxy-5-isopropyl-phenyl)-4-(4-morpholin-4-ylmethyl-phenyl)-isoxazole-3-carboxylic acid ethylamide (AUY922); and (R)-2-amino-7-[4-fluoro-2-(6-methyoxy-pyridin-2-yl)-phenyl]-4-methyl-7,8-dihydro-6H-pyrido[4,3-d]pyrimidin-5-one (HSP990).

The present invention further relates to a pharmaceutical composition comprising a compound of formula (I) or a pharmaceutically acceptable salt thereof and at least one Hsp90 inhibitor or a pharmaceutically acceptable salt thereof. In one embodiment, this pharmaceutical composition of the present invention is for use in the treatment of a proliferative disease.

The present invention further relates to the use of a pharmaceutical combination comprising a compound of formula (I) or a pharmaceutically acceptable salt thereof and at least one Hsp90 inhibitor or a pharmaceutically acceptable salt thereof, for the preparation of a medicament for the treatment of a proliferative disease.

The present invention further relates to a method for treating a proliferative disease in a subject in need thereof, comprising administering to said subject a therapeutically effective amount of a compound of formula (I) or a pharmaceutically acceptable salt thereof, and at least one Hsp90 inhibitor or a pharmaceutically acceptable salt thereof. In accordance with the present invention, the compound of formula (I) and the Hsp90 inhibitor may be administered either as a single pharmaceutical composition, as separate compositions, or sequentially.

The present invention further relates to a kit comprising a compound of formula (I) according to claim 1 or a pharmaceutically acceptable salt thereof, and at least one Hsp90 inhibitor or a pharmaceutically acceptable salt thereof.

### DESCRIPTION OF THE FIGURES

FIG. 1 shows the antitumor activity of Compound A against the PIK3CA mutant gastric cancer cell line HGC-27.

FIG. 2 shows the mean body weight of vehicle and Compound A treated groups in mice bearing the HGC-27.

For the in vivo testing in FIGS. 1 and 2, female athymic mice bearing HGC-27 subcutaneous xenografts are treated with Compound A (Cmpd A) or vehicle at the indicated doses and schedules. Treatments are started 12 days post tumor cells implantation and continue for 12 consecutive days. Statistics on change in tumor volumes are performed with a one-way ANOVA, post hoc Dunnett's (*p<0.05 vs. vehicle controls).

FIG. 3 shows the antitumor activity of vehicle, 12.5 mg/kg p.o., once a day (qd) of single agent Compound A, 50 mg/kg, i.v., twice a week (2 qw) of single agent AUY922, and the combination of Compound A with AUY922 against the PIK3CA mutant gastric cancer cell line HGC-27. Values are mean±SEM; sample size (n=10 mice per group). (*p<0.05, significant inhibition compared to vehicle control group and single agent treatment (Mann-Whitney Rank Sum Test post hoc Student Newman Kuels test).

FIG. 4 shows the mean corrected changes in body weight (represented by the ratio between body weight at day of measurement and initial body weight at day 12 [both corrected by substraction of primary tumor weight] expressed in percentage for each individual animals) of vehicle, 12.5 mg/kg compound A, 50 mg/kg AUY922 and the combination of Compound A at 25 mg/kg and AUY922 at 50 mg/kg treated groups in mice bearing the PIK3CA mutant gastric cancer cell line HGC-27.

FIG. 5 shows the antitumor activity of vehicle, 25 mg/kg p.o. qd of single agent Compound A, 50 mg/kg, iv, 2 qw of single agent AUY922, and the combination of Compound A with AUY922 against the PIK3CA mutant gastric cancer cell line HGC-27. Values are mean±SEM; sample size (n=10 mice per group). (*p<0.05, significant inhibition compared to vehicle control group and single agent treatment (Mann-Whitney Rank Sum Test post hoc Dunn's test).

FIG. 6 shows the mean corrected changes in body weight (represented by the ratio between body weight at day of measurement and initial body weight at day 12 [both corrected by substraction of primary tumor weight] expressed in percentage for each individual animals) of vehicle, 25 mg/kg compound A, 50 mg/kg AUY922 and the combination of Compound A at 25 mg/kg and AUY922 at 50 mg/kg treated groups in mice bearing the PIK3CA mutant gastric cancer cell line HGC-27.

FIG. 7 shows the antitumor activity of vehicle, 50 mg/kg p.o. qd of single agent Compound A, 50 mg/kg, iv, 2 qw of single agent AUY922, and the combination of Compound A with AUY922 against the PIK3CA mutant gastric cancer cell line HGC-27. Values are mean±SEM; sample size (n=10 mice per group). (*p<0.05, significant inhibition compared to vehicle control group and single agent treatment (Mann-Whitney Rank Sum Test post hoc Student Newman Kuels test).

FIG. 8 shows the mean corrected changes in body weight (represented by the ratio between body weight at day of measurement and initial body weight at day 12 [both corrected by substraction of primary tumor weight] expressed in percentage for each individual animals) of vehicle, 50 mg/kg compound A, 50 mg/kg AUY922 and the combination of Compound A at 50 mg/kg and AUY922 at 50 mg/kg treated groups in mice bearing the PIK3CA mutant gastric cancer cell line HGC-27.

FIG. 9 shows (a) the fractional tumor growth and (b) mean body weight changes of vehicle/placebo (n=5), 40 mg/kg p.o. qd of single agent Compound A (n=7), 50 mg/kg, iv, 2 qw of single agent AUY922 (n=8), and the combination of 40 mg/kg p.o. qd of Compound A and 50 mg/kg AUY922 (n=5) against the A375 melanoma tumor cell lines.

### DETAILED DESCRIPTION OF THE INVENTION

The following general definitions are provided to better understand the invention:

“Halogen” (or “halo”) denotes fluorine, bromine, chlorine or iodine, in particular fluorine, chlorine. Halogen-substituted groups and moieties, such as alkyl substituted by halogen (haloalkyl) can be mono-, poly- or per-halogenated.

“Hetero atoms” are atoms other than Carbon and Hydrogen, preferably nitrogen (N), oxygen (O) or sulfur (S), in particular nitrogen.

“Carbon containing groups”, moieties or molecules contain 1 to 7, preferably 1 to 6, more preferably 1 to 4, most preferably 1 or 2, carbon atoms. Any non-cyclic carbon containing group or moiety with more than 1 carbon atom is straight-chain or branched.

The prefix “lower” or “C₁-C₇” denotes a radical having up to and including a maximum of 7, especially up to and including a maximum of 4 carbon atoms, the radicals in question being either linear or branched with single or multiple branching.

“Alkyl” refers to a straight-chain or branched-chain alkyl group, preferably represents a straight-chain or branched-chain C₁₋₁₂alkyl, particularly preferably represents a straight-chain or branched-chain C₁₋₇alkyl; for example, methyl, ethyl, n- or iso-propyl, n-, iso-, sec- or tert-butyl, n-pentyl, n-hexyl, n-heptyl, n-octyl, n-nonyl, n-decyl, n-undecyl, n-dodecyl, with particular preference given to methyl, ethyl, n-propyl, iso-propyl and n-butyl and iso-butyl. Alkyl may be unsubstituted or substituted. Exemplary substituents include, but are not limited to deuterium, hydroxy, alkoxy, halo and amino. An example of a substituted alkyl is trifluoromethyl. Cycloalkyl may also be a substituent to alkyl. An example of such a case is the moiety (alkyl)-cyclopropyl or alkandiyl-cycloproyl, e.g. —CH₂-cyclopropyl. C₁-C₇-alkyl is preferably alkyl with from and including 1 up to and including 7, preferably from and including 1 to and including 4, and is linear or branched; preferably, lower alkyl is butyl, such as n-butyl, sec-butyl, isobutyl, tert-butyl, propyl, such as n-propyl or isopropyl, ethyl or preferably methyl.

Each alkyl part of other groups like “alkoxy”, “alkoxyalkyl”, “alkoxycarbonyl”, “alkoxy-carbonylalkyl”, “alkylsulfonyl”, “alkylsulfoxyl”, “alkylamino”, “haloalkyl” shall have the same meaning as described in the above-mentioned definition of “alkyl”.

“Alkandiyl” refers to a straight-chain or branched-chain alkandiyl group bound by two different Carbon atoms to the moiety, it preferably represents a straight-chain or branched-chain C₁₋₁₂ alkandiyl, particularly preferably represents a straight-chain or branched-chain C₁₋₆ alkandiyl; for example, methandiyl (—CH₂—), 1,2-ethanediyl (—CH₂—CH₂—), 1,1-ethanediyl ((—CH(CH₃)—), 1,1-, 1,2-, 1,3-propanediyl and 1,1-, 1,2-, 1,3-, 1,4-butanediyl, with particular preference given to methandiyl, 1,1-ethanediyl, 1,2-ethanediyl, 1,3-propanediyl, 1,4-butanediyl.

“Alkendiyl” refers to a straight-chain or branched-chain alkendiyl group bound by two different Carbon atoms to the molecule, it preferably represents a straight-chain or branched-chain C₂₋₆ alkandiyl; for example, —CH═CH—, —CH═C(CH₃)—, —CH═CH—CH₂—, —C(CH₃)═CH—CH₂—, —CH═C(CH₃)—CH₂—, —CH═CH—C(CH₃)H—, —CH═CH—CH═CH—, —C(CH₃)═CH—CH═CH—, —CH═C(CH₃)—CH═CH—, with particular preference given to —CH═CH—CH₂—, —CH═CH—CH═CH—. Alkendiyl may be substituted or unsubstituted

“Cycloalkyl” refers to a saturated or partially saturated, monocyclic, fused polycyclic, or Spiro polycyclic, carbocycle having from 3 to 12 ring atoms per carbocycle. Illustrative examples of cycloalkyl groups include the following moieties: cyclopropyl, cyclobutyl, cyclpentyl and cyclohexyl. Cycloalkyl may be unsubstituted or substituted; exemplary substituents are provided in the definition for alkyl and also include alkyl itself (e.g. methyl). A moiety like —(CH₃)cyclopropyl is considered substituted cycloalkyl.

“Aryl” refers to an aromatic homocyclic ring system (i.e. only Carbon as ring forming atoms) with 6 or more carbon atoms; aryl is preferably an aromatic moiety with 6 to 14 ring carbon atoms, more preferably with 6 to 10 ring carbon atoms, such as phenyl or naphthyl, preferably phenyl. Aryl may be unsubstituted or substituted by one or more, preferably up to three, more preferably up to two substituents independently selected from the group consisting of unsubstituted or substituted heterocyclyl as described below, especially pyrrolidinyl, such as pyrrolidino, oxopyrrolidinyl, such as oxopyrrolidino, C₁-C₇-alkyl-pyrrolidinyl, 2,5-di-(C₁-C₇alkyl)pyrrolidinyl, such as 2,5-di-(C₁-C₇alkyl)-pyrrolidino, tetrahydrofuranyl, thiophenyl, C₁-C₇-alkylpyrazolidinyl, pyridinyl, C₁-C₇-alkylpiperidinyl, piperidino, piperidino substituted by amino or N-mono- or N,N-di-[lower alkyl, phenyl, C₁-C₇-alkanoyl and/or phenyl-lower alkyl)-amino, unsubstituted or N-lower alkyl substituted piperidinyl bound via a ring carbon atom, piperazino, lower alkylpiperazino, morpholino, thiomorpholino, S-oxo-thiomorpholino or S,S-dioxothiomorpholino; C₁-C₇-alkyl, amino-C₁-C₇-alkyl, N—C₁-C₇-alkanoylamino-C₁-C₇-alkyl, N—C₁-C₇-alkanesulfonyl-amino-C₁-C₇-alkyl, carbamoyl-C₁-C₇-alkyl, [N-mono- or N,N-di-(C₁-C₇-alkyl)-carbamoyl]-C₁-C₇-alkyl, C₁-C₇-alkanesulfinyl-C₁-C₇-alkyl, C₁-C₇-alkanesulfonyl-C₁-C₇-alkyl, phenyl, naphthyl, mono- to tri-[C₁-C₇-alkyl, halo and/or cyano]-phenyl or mono- to tri-[C₁-C₇-alkyl, halo and/or cyano]-naphthyl; C₃-C₈-cycloalkyl, mono- to tri-[C₁-C₇-alkyl and/or hydroxy]-C₃-C₈-cycloalkyl; halo, hydroxy, lower alkoxy, lower-alkoxy-lower alkoxy, (lower-alkoxy)-lower alkoxy-lower alkoxy, halo-C₁-C₇-alkoxy, phenoxy, naphthyloxy, phenyl- or naphthyl-lower alkoxy; amino-C₁-C₇alkoxy, lower-alkanoyloxy, benzoyloxy, naphthoyloxy, formyl (CHO), amino, N-mono- or N,N-di-(C₁-C₇-alkyl)-amino, C₁-C₇-alkanoylamino, C₁-C₇-alkanesulfonylamino, carboxy, lower alkoxy carbonyl, e.g.; phenyl- or naphthyl-lower alkoxycarbonyl, such as benzyloxycarbonyl; C₁-C₇-alkanoyl, such as acetyl, benzoyl, naphthoyl, carbamoyl, N-mono- or N,N-disubstituted carbamoyl, such as N-mono- or N,N-di-substituted carbamoyl wherein the substitutents are selected from lower alkyl, (lower-alkoxy)-lower alkyl and hydroxy-lower alkyl; amidino, guanidino, ureido, mercapto, lower alkylthio, phenyl- or naphthylthio, phenyl- or naphthyl-lower alkylthio, lower alkyl-phenylthio, lower alkyl-naphthylthio, halo-lower alkylmercapto, sulfo (—SO₃H), lower alkanesulfonyl, phenyl- or naphthyl-sulfonyl, phenyl- or naphthyl-lower alkylsulfonyl, alkylphenyl-sulfonyl, halo-lower alkylsulfonyl, such as trifluoromethanesulfonyl; sulfonamido, benzosulfonamido, azido, azido-C₁-C₇-alkyl, especially azidomethyl, C₁-C₇-alkanesulfonyl, sulfamoyl, N-mono- or N,N-di-(C₁-C₇-alkyl)-sulfamoyl, morpholinosulfonyl, thiomorpho-linosulfonyl, cyano and nitro; where each phenyl or naphthyl (also in phenoxy or naphthoxy) mentioned above as substituent or part of a substituent of substituted alkyl (or also of substituted aryl, heterocyclyl etc. mentioned herein) is itself unsubstituted or substituted by one or more, e.g. up to three, preferably 1 or 2, substituents independently selected from halo, halo-lower alkyl, such as trifluoromethyl, hydroxy, lower alkoxy, azido, amino, N-mono- or N,N-di-(lower alkyl and/or C₁-C₇-alkanoyl)-amino, nitro, carboxy, lower-alkoxycarbonyl, carbamoyl, cyano and/or sulfamoyl.

“Heterocyclyl” refers to a heterocyclic radical that is unsaturated (=carrying the highest possible number of conjugated double bonds in the ring(s)), saturated or partially saturated and is preferably a monocyclic or in a broader aspect of the invention bicyclic, tricyclic or spirocyclic ring; and has 3 to 24, more preferably 4 to 16, most preferably 5 to 10 and most preferably 5 or 6 ring atoms; wherein one or more, preferably one to four, especially one or two ring atoms are a heteroatom (the remaining ring atoms therefore being carbon). The bonding ring (i.e. the ring connecting to the molecule) preferably has 4 to 12, especially 5 to 7 ring atoms. The term heterocyclyl also includes heteroaryl. The heterocyclic radical (heterocyclyl) may be unsubstituted or substituted by one or more, especially 1 to 3, substituents independently selected from the group consisting of the substituents defined above for substituted alkyl and/or from one or more of the following substituents: oxo (═O), thiocarbonyl (═S), imino(═NH), imino-lower alkyl. Further, heterocyclyl is especially a heterocyclyl radical selected from the group consisting of oxiranyl, azirinyl, aziridinyl, 1,2-oxathiolanyl, thienyl (=thiophenyl), furanyl, tetrahydrofuryl, pyranyl, thiopyranyl, thianthrenyl, isobenzofuranyl, benzofuranyl, chromenyl, 2H-pyrrolyl, pyrrolyl, pyrrolinyl, pyrrolidinyl, imidazolyl, imidazolidinyl, benzimidazolyl, pyrazolyl, pyrazinyl, pyrazolidinyl, thiazolyl, isothiazolyl, dithiazolyl, oxazolyl, isoxazolyl, pyridyl, pyrazinyl, pyrimidinyl, piperidinyl, piperazinyl, pyridazinyl, morpholinyl, thiomorpholinyl, (S-oxo or S,S-dioxo)-thiomorpholinyl, indolizinyl, azepanyl, diazepanyl, especially 1,4-diazepanyl, isoindolyl, 3H-indolyl, indolyl, benzimidazolyl, cumaryl, indazolyl, triazolyl, tetrazolyl, purinyl, 4H-quinoli-zinyl, isoquinolyl, quinolyl, tetrahydroquinolyl, tetrahydroisoquinolyl, decahydroquinolyl, octahydroisoquinolyl, benzofuranyl, dibenzofuranyl, benzothiophenyl, dibenzothiophenyl, phthalazinyl, naphthyridinyl, quinoxalyl, quinazolinyl, quinazolinyl, cinnolinyl, pteridinyl, carbazolyl, beta-carbolinyl, phenanthridinyl, acridinyl, perimidinyl, phenanthrolinyl, furazanyl, phenazinyl, phenothiazinyl, phenoxazinyl, chromenyl, isochromanyl, chromanyl, benzo[1,3]di-oxol-5-yl and 2,3-dihydro-benzo[1,4]dioxin-6-yl, each of these radicals being unsubstituted or substituted by one or more, preferably up to three, substituents selected from those mentioned above for substituted aryl and/or from one or more of the following substituents: oxo (═O), thiocarbonyl (═S), imino(═NH), imino-lower alkyl.

“Arylalkyl” refers to an aryl group bound to the molecule via an alkyl group, such as a methyl or ethyl group, preferably phenethyl or benzyl, in particular benzyl. Similarly, cycloalkyl-alkyl and heterocyclyl-alkyl represents a cycloalkyl group bound to the molecule via an alkyl group or a heterocyclyl group bound to the molecule via an alkyl group. In each instance, aryl, heterocyclyl, cycloalkyl and alkyl may be substituted as defined above.

“Salts” (which, what is meant by “or salts thereof” or “or a salt thereof”), can be present alone or in mixture with free compound, e.g. the compound of the formula (I), and are preferably pharmaceutically acceptable salts. Such salts of the compounds of formula (I) are formed, for example, as acid addition salts, preferably with organic or inorganic acids, from compounds of formula (I) with a basic nitrogen atom. Suitable inorganic acids are, for example, halogen acids, such as hydrochloric acid, sulfuric acid, or phosphoric acid. Suitable organic acids are, e.g., carboxylic acids or sulfonic acids, such as fumaric acid or methansulfonic acid. For isolation or purification purposes it is also possible to use pharmaceutically unacceptable salts, for example picrates or perchlorates. For therapeutic use, only pharmaceutically acceptable salts or free compounds are employed (where applicable in the form of pharmaceutical preparations), and these are therefore preferred. In view of the close relationship between the novel compounds in free form and those in the form of their salts, including those salts that can be used as intermediates, for example in the purification or identification of the novel compounds, any reference to the free compounds hereinbefore and hereinafter is to be understood as referring also to the corresponding salts, as appropriate and expedient. The salts of compounds of formula (I) are preferably pharmaceutically acceptable salts; suitable counter-ions forming pharmaceutically acceptable salts are known in the field.

“Combination” refers to either a fixed combination in one dosage unit form, or a non-fixed combination (or kit of parts) for the combined administration where a compound of the formula (I) and a combination partner (e.g. another drug as explained below, also referred to as “therapeutic agent” or “co-agent”) may be administered independently at the same time or separately within time intervals, especially where these time intervals allow that the combination partners show a cooperative, e.g. synergistic effect. The term “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “fixed combination” means that the active ingredients, e.g. a compound of formula (I) and a combination partner, are both administered to a patient simultaneously in the form of a single entity or dosage. The terms “non-fixed combination” or “kit of parts” mean that the active ingredients, e.g. a compound of formula (I) and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active ingredients.

“Treatment” includes prophylactic (preventive) and therapeutic treatment as well as the delay of progression of a disease or disorder. The term “prophylactic” means the prevention of the onset or recurrence of diseases involving proliferative diseases. The term “delay of progression” as used herein means administration of the combination to patients being in a pre-stage or in an early phase of the proliferative disease to be treated, in which patients for example a pre-form of the corresponding disease is diagnosed or which patients are in a condition, e.g. during a medical treatment or a condition resulting from an accident, under which it is likely that a corresponding disease will develop.

“Subject” is intended to include animals. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or potentially capable of suffering from a brain tumor disease. Particularly preferred, the subject is human.

“Pharmaceutical preparation” or “pharmaceutical composition” refer to a mixture or solution containing at least one therapeutic compound to be administered to a mammal, e.g., a human in order to prevent, treat or control a particular disease or condition affecting the mammal.

“Co-administer”, “co-administration” or “combined administration” or the like are meant to encompass administration of the selected therapeutic agents to a single patient, and are intended to include treatment regimens in which the agents are not necessarily administered by the same route of administration or at the same time.

“Pharmaceutically acceptable” refers to those compounds, materials, compositions and/or dosage forms, which are, within the scope of sound medical judgment, suitable for contact with the tissues of mammals, especially humans, without excessive toxicity, irritation, allergic response and other problem complications commensurate with a reasonable benefit/risk ratio.

“Therapeutically effective” preferably relates to an amount that is therapeutically or in a broader sense also prophylactically effective against the progression of a proliferative disease.

“Single pharmaceutical composition” refers to a single carrier or vehicle formulated to deliver effective amounts of both therapeutic agents to a patient. The single vehicle is designed to deliver an effective amount of each of the agents, along with any pharmaceutically acceptable carriers or excipients. In some embodiments, the vehicle is a tablet, capsule, pill, or a patch. In other embodiments, the vehicle is a solution or a suspension.

“Dose range” refers to an upper and a lower limit of an acceptable variation of the amount of agent specified. Typically, a dose of the agent in any amount within the specified range can be administered to patients undergoing treatment.

The terms “about” or “approximately” usually means within 20%, more preferably within 10%, and most preferably still within 5% of a given value or range. Alternatively, especially in biological systems, the term “about” means within about a log (i.e., an order of magnitude) preferably within a factor of two of a given value.

The present invention relates to a pharmaceutical combination comprising (a) a compound of formula (I), as defined below, or a pharmaceutically acceptable salt thereof; and (b) at least one Hsp90 inhibitor or a pharmaceutically acceptable salt thereof. Such combination may be for simultaneous, separate or sequential use for the treatment of a proliferative disease.

Specific 2-carboxamide cycloamino urea derivatives which are suitable for the present invention, their preparation and suitable pharmaceutical formulations containing the same are described in WO 2010/029082 and include compounds of formula (I)

[chemical structure]

wherein
A represents a heteroaryl selected from the group consisting of:

[chemical structure]

R¹ represents one of the following substituents: (1) unsubstituted or substituted, preferably substituted C₁-C₇-alkyl, wherein said substituents are independently selected from one or more, preferably one to nine of the following moieties: deuterium, fluoro, or one to two of the following moieties C₃-C₅-cycloalkyl; (2) optionally substituted C₃-C₅-cycloalkyl wherein said substituents are independently selected from one or more, preferably one to four of the following moieties: deuterium, C₁-C₄-alkyl (preferably methyl), fluoro, cyano, aminocarbonyl; (3) optionally substituted phenyl wherein said substituents are independently selected from one or more, preferably one to two of the following moieties: deuterium, halo, cyano, C₁-C₇-alkylamino, di(C₃-C₇-alkyl)amino, C₁-C₇-alkylaminocarbonyl, di(C₁-C₇-alkyl)aminocarbonyl, C₁-C₇-alkoxy; (4) optionally mono- or di-substituted amine; wherein said substituents are independently selected from the following moieties: deuterium, C₁-C₇-alkyl (which is unsubstituted or substituted by one or more substituents selected from the group of deuterium, fluoro, chloro, hydroxy), phenylsulfonyl (which is unsubstituted or substituted by one or more, preferably one, C₁-C₇-alkyl, C₁-C₇-alkoxy, di(C₁-C₇-alkyl)amino-C₁-C₇-alkoxy); (5) substituted sulfonyl; wherein said substituent is selected from the following moieties: C₁-C₇-alkyl (which is unsubstituted or substituted by one or more substituents selected from the group of deuterium, fluoro), pyrrolidino, (which is unsubstituted or substituted by one or more substituents selected from the group of deuterium, hydroxy, oxo; particularly one oxo); (6) fluoro, chloro;
R² represents hydrogen;
R³ represents (1) hydrogen, (2) fluoro, chloro, (3) optionally substituted methyl, wherein said substituents are independently selected from one or more, preferably one to three of the following moieties: deuterium, fluoro, chloro, dimethylamino;
with the exception of (S)-Pyrrolidine-1,2-dicarboxylic acid 2-amide 1-({5-[2-(tert-butyl)-pyrimidin-4-yl]-4-methyl-thiazol-2-yl}-amide).
   The radicals and symbols as used in the definition of a compound of formula (I) have the meanings as disclosed in WO 2010/029082 which is hereby incorporated by reference in its entirety.

As disclosed in WO2010/029082, these 2-carboxamide cycloamino urea derivative compounds of formula (I) have been found to have significant inhibitory activity for phosphatidylinositol 3-kinases (or PI3K). These compounds of formula (I) have advantageous pharmacological properties as a PI3K inhibitor and show a high selectivity for the PI3-kinase alpha subtype as compared to the beta and/or delta and/or gamma subtypes.

A preferred compound of formula (I) for the present invention is a compound which is specifically described in WO2010/029082. A very preferred compound of the present invention is (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (Compound A) or a pharmaceutically acceptable salt thereof. The synthesis of (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) is described in WO2010/029082 as Example 15.

Pharmaceutical combinations of the present invention include at least one compound targeting, decreasing or inhibiting the intrinsic ATPase activity of Hsp90 and/or degrading, targeting, decreasing or inhibiting the Hsp90 client proteins via the ubiquitin proteosome pathway. Such compounds will be referred to as “Heat shock protein 90 inhibitors” or “Hsp90 inhibitors”.

Suitable Hsp90 inhibitors include, but are not limited to,
(a) the geldanamycin derivative, Tanespimycin (17-allylamino-17-demethoxygeldanamycin)(also known as KOS-953 and 17-AAG), which is available from Sigma-Aldrich Co, LLC (St. Louis, Mo.), and disclosed in U.S. Pat. No. 4,261,989, dated Apr. 14, 1981, which is hereby incorporated into the present application by reference, and other geldanamycin-related compounds;
(b) Radicicol, which is available from Sigma-Aldrich Co, LLC (St. Louis, Mo.);
(c) 6-Chloro-9-(4-methoxy-3,5-dimethylpyridin-2-ylmethyl)-9H-purin-2-amine methanesulfonate (also known as CNF2024)(Conforma Therapeutics Corp.);
(d) IPI504;
(e) SNX5422;
(f) 5-(2,4-Dihydroxy-5-isopropyl-phenyl)-4-(4-morpholin-4-ylmethyl-phenyl)-isoxazole-3-carboxylic acid ethylamide (AUY922), which is disclosed in structure and with the process for its manufacture in PCT Application No. WO04/072051, published on Aug. 26, 2004, which is hereby incorporated into the present application by reference; and
(g) (R)-2-amino-7-[4-fluoro-2-(6-methyoxy-pyridin-2-yl)-phenyl]-4-methyl-7,8-dihydro-6H-pyrido[4,3-d]pyrimidin-5-one (HSP990), which is disclosed in structure and with the process for its manufacture in U.S. Patent Application Publication No. 2007-0123546, published on May 31, 2007, which is hereby incorporated into the present application by reference;
and pharmaceutically acceptable salts thereof.

Preferred Hsp90 inhibitors for the present invention are 5-(2,4-Dihydroxy-5-isopropyl-phenyl)-4-(4-morpholin-4-ylmethyl-phenyl)-isoxazole-3-carboxylic acid ethylamide (AUY922) and (R)-2-amino-7-[4-fluoro-2-(6-methyoxy-pyridin-2-yl)-phenyl]-4-methyl-7,8-dihydro-6H-pyrido[4,3-d]pyrimidin-5-one (HSP990) or pharmaceutically acceptable salts thereof.

Comprised are likewise the pharmaceutically acceptable salts thereof, the corresponding racemates, diastereoisomers, enantiomers, tautomers, as well as the corresponding crystal modifications of above disclosed compounds where present, e.g. solvates, hydrates and polymorphs, which are disclosed therein. The compounds used as active ingredients in the combinations of the present invention can be prepared and administered as described in the cited documents, respectively. Also within the scope of this invention is the combination of more than two separate active ingredients as set forth above, i.e., a pharmaceutical combination within the scope of this invention could include three active ingredients or more.

In one embodiment of the present invention, the pharmaceutical combination comprises the compound of formula (I) that is (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or a pharmaceutically acceptable salt thereof, and at least one Hsp90 inhibitor selected from 5-(2,4-Dihydroxy-5-isopropyl-phenyl)-4-(4-morpholin-4-ylmethyl-phenyl)-isoxazole-3-carboxylic acid ethylamide (AUY922), (R)-2-amino-7-[4-fluoro-2-(6-methyoxy-pyridin-2-yl)-phenyl]-4-methyl-7,8-dihydro-6H-pyrido[4,3-d]pyrimidin-5-one (HSP990), or pharmaceutically acceptable salts thereof.

In one embodiment of the present invention, the pharmaceutical combination comprises the compound of formula (I) that is (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or pharmaceutically acceptable salts thereof, and at least one Hsp90 inhibitor 5-(2,4-Dihydroxy-5-isopropyl-phenyl)-4-(4-morpholin-4-ylmethyl-phenyl)-isoxazole-3-carboxylic acid ethylamide (AUY922) or a pharmaceutically acceptable salt thereof.

It has now been surprisingly found that the combination of a compound of formula (I), which is a alpha-specific PI3K inhibitor, and at least one Hsp90 inhibitor possess beneficial therapeutic properties, which render it particularly useful for the treatment of proliferative diseases, particularly cancer.

In one aspect, the present invention provides a pharmaceutical combination comprising (a) a compound of formula (I), particularly the compound (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide), or a pharmaceutically acceptable salt thereof, and (b) at least one Hsp90 inhibitor or a pharmaceutically acceptable salt thereof, for use in the treatment of a proliferative disease, particularly cancer.

In one aspect, the present invention provides the use of a pharmaceutical combination comprising a compound of formula (I) or a pharmaceutically acceptable salt thereof and at least one Hsp90 inhibitor or a pharmaceutically acceptable salt thereof, for the preparation of a medicament for the treatment of a proliferative disease.

In one aspect, the present invention further relates to a method for treating a proliferative disease in a subject in need thereof, comprising administering to said subject a therapeutically effective amount of a compound of formula (I) or a pharmaceutically acceptable salt thereof, and at least one Hsp90 inhibitor or a pharmaceutically acceptable salt thereof. In accordance with the present invention, the compound of formula (I) and the Hsp90 inhibitor may be administered either as a single pharmaceutical composition, as separate compositions, or sequentially.

Preferably, the present invention is useful for the treating a mammal, especially humans, suffering from a proliferative disease such as cancer.

To demonstrate that the combination of a compound of formula (I) and at least one Hsp90 inhibitor is particularly suitable for the effective treatment of proliferative diseases with good therapeutic margin and other advantages, clinical trials can be carried out in a manner known to the skilled person.

Suitable clinical studies are, e.g., open label, dose escalation studies in patients with proliferative diseases. Such studies prove in particular the synergism of the active ingredients of the combination of the invention. The beneficial effects can be determined directly through the results of these studies which are known as such to a person skilled in the art. Such studies are, in particular, suitable to compare the effects of a monotherapy using the active ingredients and a combination of the invention. Preferably, the dose of agent (a) is escalated until the Maximum Tolerated Dosage is reached, and agent (b) is administered with a fixed dose. Alternatively, the agent (a) is administered in a fixed dose and the dose of agent (b) is escalated. Each patient receives doses of the agent (a) either daily or intermittent. The efficacy of the treatment can be determined in such studies, e.g., after 12, 18 or 24 weeks by evaluation of symptom scores every 6 weeks.

The administration of a pharmaceutical combination of the invention results not only in a beneficial effect, e.g., a synergistic therapeutic effect, e.g., with regard to alleviating, delaying progression of or inhibiting the symptoms, but also in further surprising beneficial effects, e.g., fewer side effects, an improved quality of life or a decreased morbidity, compared with a monotherapy applying only one of agents (a) or agents (b) used in the combination of the invention.

A further benefit is that lower doses of the active ingredients of the combination of the invention can be used, e.g., that the dosages need not only often be smaller but are also applied less frequently, which may diminish the incidence or severity of side effects. This is in accordance with the desires and requirements of the patients to be treated.

It is one objective of this invention to provide a pharmaceutical composition comprising a quantity, which is jointly therapeutically effective at targeting or preventing proliferative diseases, of each combination partner agent (a) and (b) of the invention. In one aspect, the present invention relates to a pharmaceutical composition comprising a compound of formula (I) or a pharmaceutically acceptable salt thereof and at least one Hsp90 inhibitor or a pharmaceutically acceptable salt thereof. In one embodiment, such pharmaceutical composition of the present invention is for use in the treatment of a proliferative disease. In accordance with the present invention, agent (a) and agent (b) may be administered together in a single pharmaceutical composition, separately in one combined unit dosage form or in two separate unit dosage forms, or sequentially. The unit dosage form may also be a fixed combination.

The pharmaceutical compositions for separate administration of agent (a) and agent (b) or for the administration in a fixed combination (i.e., a single galenical composition comprising at least two combination partners (a) and (b)) according to the invention may be prepared in a manner known per se and are those suitable for enteral, such as oral or rectal, topical, and parenteral administration to subjects, including mammals (warm-blooded animals) such as humans, comprising a therapeutically effective amount of at least one pharmacologically active combination partner alone, e.g., as indicated above, or in combination with one or more pharmaceutically acceptable carriers or diluents, especially suitable for enteral or parenteral application. Suitable pharmaceutical compositions contain, e.g., from about 0.1% to about 99.9%, preferably from about 1% to about 60%, of the active ingredient(s).

Pharmaceutical compositions for the combination therapy for enteral or parenteral administration are, e.g., those in unit dosage forms, such as sugar-coated tablets, tablets, capsules or suppositories, ampoules, injectable solutions or injectable suspensions. Topical administration is e.g. to the skin or the eye, e.g. in the form of lotions, gels, ointments or creams, or in a nasal or a suppository form. If not indicated otherwise, these are prepared in a manner known per se, e.g., by means of conventional mixing, granulating, sugar-coating, dissolving or lyophilizing processes. It will be appreciated that the unit content of agent (a) or agent (b) contained in an individual dose of each dosage form need not in itself constitute an effective amount since the necessary effective amount can be reached by administration of a plurality of dosage units.

Pharmaceutical compositions may comprise one or more pharmaceutical acceptable carriers or diluents and may be manufactured in conventional manner by mixing one or both combination partners with a pharmaceutically acceptable carrier or diluent. Examples of pharmaceutically acceptable diluents include, but are not limited to, lactose, dextrose, mannitol, and/or glycerol, and/or lubricants and/or polyethylene glycol. Examples of pharmaceutically acceptable binders include, but are not limited to, magnesium aluminum silicate, starches, such as corn, wheat or rice starch, gelatin, methylcellulose, sodium carboxymethylcellulose and/or polyvinylpyrrolidone, and, if desired, pharmaceutically acceptable disintegrators include, but are not limited to, starches, agar, alginic acid or a salt thereof, such as sodium alginate, and/or effervescent mixtures, or adsorbents, dyes, flavorings and sweeteners. It is also possible to use the compounds of the present invention in the form of parenterally administrable compositions or in the form of infusion solutions. The pharmaceutical compositions may be sterilized and/or may comprise excipients, for example preservatives, stabilizers, wetting compounds and/or emulsifiers, solubilisers, salts for regulating the osmotic pressure and/or buffers.

In particular, a therapeutically effective amount of each of the combination partner of the combination of the invention may be administered simultaneously or sequentially and in any order, and the components may be administered separately or as a fixed combination. For example, the method of preventing or treating proliferative diseases according to the invention may comprise: (i) administration of the first agent (a) in free or pharmaceutically acceptable salt form; and (ii) administration of an agent (b) in free or pharmaceutically acceptable salt form, simultaneously or sequentially in any order, in jointly therapeutically effective amounts, preferably in synergistically effective amounts, e.g., in daily or intermittently dosages corresponding to the amounts described herein. The individual combination partners of the combination of the invention may be administered separately at different times during the course of therapy or concurrently in divided or single combination forms. Furthermore, the term administering also encompasses the use of a pro-drug of a combination partner that convert in vivo to the combination partner as such. The instant invention is therefore to be understood as embracing all such regimens of simultaneous or alternating treatment and the term “administering” is to be interpreted accordingly.

The effective dosage of each of combination partner agent (a) or agent (b) employed in the combination of the invention may vary depending on the particular compound or pharmaceutical composition employed, the mode of administration, the condition being treated, the severity of the condition being treated. Thus, the dosage regimen of the combination of the invention is selected in accordance with a variety of factors including type, species, age, weight, sex and medical condition of the patient; the severity of the condition to be treated; the route of administration; the renal and hepatic function of the patient; and the particular compound employed. A physician, clinician or veterinarian of ordinary skill can readily determine and prescribe the effective amount of the drug required to prevent, counter or arrest the progress of the condition. Optimal precision in achieving concentration of drug within the range that yields efficacy requires a regimen based on the kinetics of the drug's availability to target sites. This involves a consideration of the distribution, equilibrium, and elimination of a drug.

For purposes of the present invention, a therapeutically effective dose will generally be a total daily dose administered to a host in single or divided doses. The compound of formula (I) may be administered to a host in a daily dosage range of, for example, from about 0.05 to about 50 mg/kg body weight of the recipient, preferably about 0.1-25 mg/kg body weight of the recipient, more preferably from about 0.5 to 10 mg/kg body weight of the recipient. For administration to a 70 kg person, the dosage range of the compound of formula (I) would most preferably be about 35-700 mg daily. Agent (b) may be administered to a host in a daily dosage range of, for example, from about 0.001 to 1000 mg/kg body weight of the recipient and more preferred from 1.0 to 30 mg/kg body weight of the recipient. Dosage unit compositions may contain such amounts of submultiples thereof to make up the daily dose.

A further benefit is that lower doses of the active ingredients of the combination of the invention can be used, e.g., that the dosages need not only often be smaller but are also applied less frequently, or can be used in order to diminish the incidence of side effects. This is in accordance with the desires and requirements of the patients to be treated.

The combination of the compound of formula (I) and an HSP90 inhibitor can be used alone or combined with at least one other pharmaceutically active compound for use in these pathologies. These active compounds can be combined in the same pharmaceutical preparation or in the form of combined preparations “kit of parts” in the sense that the combination partners can be dosed independently or by use of different fixed combinations with distinguished amounts of the combination partners, i.e., simultaneously or at different time points. The parts of the kit of parts can then, e.g., be administered simultaneously or chronologically staggered, that is at different time points and with equal or different time intervals for any part of the kit of parts. Non-limiting examples of compounds which can be cited for use in combination with the combination of a compound of formula (I) and at least one HSP90 inhibitor are cytotoxic chemotherapy drugs, such as anastrozole, doxorubicin hydrochloride, flutamide, dexamethaxone, docetaxel, cisplatin, paclitaxel, etc. Further, the combination of a pyrimidylaminobenzamide compound and an HSP90 inhibitor could be combined with other inhibitors of signal transduction or other oncogene-targeted drugs with the expectation that significant synergy would result.

The combination of the present invention is particularly useful for the treatment of proliferative diseases. The term “proliferative disease” includes, but not restricted to, cancer, tumor, hyperplasia, restenosis, cardiac hypertrophy, immune disorder and inflammation.

Examples for a proliferative disease the can be treated with the combination of the present invention are for instance cancers, including, for example, sarcoma; lung; bronchus; prostate; breast (including sporadic breast cancers and sufferers of Cowden disease); pancreas; gastrointestinal cancer or gastric; colon; rectum; colorectal adenoma; thyroid; liver; intrahepatic bile duct; hepatocellular; adrenal gland; stomach; glioma; glioblastoma; endometrial; kidney; renal pelvis; urinary bladder; uterine corpus; uterine cervix; vagina; ovary; multiple myeloma; esophagus; a leukaemia; acute myelogenous leukemia; chronic myelogenous leukemia; lymphocytic leukemia; myeloid leukemia; brain; oral cavity and pharynx; larynx; small intestine; non-Hodgkin lymphoma; melanoma; villous colon adenoma; a neoplasia; a neoplasia of epithelial character; lymphomas; a mammary carcinoma; basal cell carcinoma; squamous cell carcinoma; actinic keratosis; a tumor of the neck or head; polycythemia vera; essential thrombocythemia; myelofibrosis with myeloid metaplasia; and Walden stroem disease.

Further examples include, polycythemia vera, essential thrombocythemia, myelofibrosis with myeloid metaplasia, asthma, COPD, ARDS, Loffler's syndrome, eosinophilic pneumonia, parasitic (in particular metazoan) infestation (including tropical eosinophilia), bronchopulmonary aspergillosis, polyarteritis nodosa (including Churg-Strauss syndrome), eosinophilic granuloma, eosinophil-related disorders affecting the airways occasioned by drug-reaction, psoriasis, contact dermatitis, atopic dermatitis, alopecia greata, erythema multiforme, dermatitis herpetiformis, scleroderma, vitiligo, hypersensitivity angiitis, urticaria, bullous pemphigoid, lupus erythematosus, pemphisus, epidermolysis bullosa acquisita, autoimmune haematogical disorders (e.g. haemolytic anaemia, aplastic anaemia, pure red cell anaemia and idiopathic thrombocytopenia), systemic lupus erythematosus, polychondritis, scleroderma, Wegener granulomatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine opthalmopathy, Grave's disease, sarcoidosis, alveolitis, chronic hypersensitivity pneumonitis, multiple sclerosis, primary biliary cirrhosis, uveitis (anterior and posterior), interstitial lung fibrosis, psoriatic arthritis, glomerulonephritis, cardiovascular diseases, atherosclerosis, hypertension, deep venous thrombosis, stroke, myocardial infarction, unstable angina, thromboembolism, pulmonary embolism, thrombolytic diseases, acute arterial ischemia, peripheral thrombotic occlusions, and coronary artery disease, reperfusion injuries, retinopathy, such as diabetic retinopathy or hyperbaric oxygen-induced retinopathy, and conditions characterized by elevated intraocular pressure or secretion of ocular aqueous humor, such as glaucoma.

In one embodiment, the proliferative disease treated by the combination of the present invention is a cancer that can be beneficially treated by the inhibition of HSP90 and/or PI3K including, for example, gastric, lung and bronchus; prostate; breast; pancreas; colon; rectum; thyroid; liver and intrahepatic bile duct; kidney and renal pelvis; urinary bladder; uterine corpus; uterine cervix; ovary; multiple myeloma; esophagus; acute myelogenous leukemia; chronic myelogenous leukemia; lymphocytic leukemia; myeloid leukemia; brain; oral cavity and pharynx; larynx; small intestine; non-Hodgkin lymphoma; melanoma; and villous colon adenoma.

In one embodiment, the proliferative disease treated by the combination of the present invention is a cancer of the esophagus, gastrointestinal cancer or gastric.

Where a tumor, a tumor disease, sarcoma, a carcinoma or a cancer are mentioned, also metastasis in the original organ or tissue and/or in any other location are implied alternatively or in addition, whatever the location of the tumor and/or metastasis.

The combination of the present invention is particularly useful for the treatment of proliferative diseases, particularly cancers and other malignancies, mediated by phosphatidylinositol 3-kinase (PI3K), particularly the alpha-subunit of PI3K, and/or Hsp90 (or those depending from PI3K or Hsp90). Proliferative diseases may include those showing overexpression or amplification of PI3K alpha, somatic mutation of PIK3CA or germline mutations or somatic mutation of PTEN or mutations and translocation of p85α that serve to up-regulate the p85-p110 complex.

In one embodiment, the present invention relates to a method for treating a proliferative disorder comprising administering to said subject a therapeutically effective amount of a compound of formula (I) selected from (S)-Pyrrolidine-1,2-dicarboxylic acid 2-amide {1-({4-methyl-5-[2-(2,2,2-trifluoro-1,1-dimethyl-ethyl)-pyridin-4-yl]-thiazol-2-yl}-amide) (Compound A) or a pharmaceutically acceptable salt thereof, and at least one Hsp90 inhibitor selected from the geldanamycin derivative, Tanespimycin (17-allylamino-17-demethoxygeldanamycin) (also known as KOS-953 and 17-MG); Radicicol; 6-Chloro-9-(4-methoxy-3,5-dimethylpyridin-2-ylmethyl)-9H-purin-2-amine methanesulfonate (also known as CNF2024); IPI504; SNX5422; 5-(2,4-Dihydroxy-5-isopropyl-phenyl)-4-(4-morpholin-4-ylmethyl-phenyl)-isoxazole-3-carboxylic acid ethylamide (AUY922); and (R)-2-amino-7-[4-fluoro-2-(6-methyoxy-pyridin-2-yl)-phenyl]-4-methyl-7,8-dihydro-6H-pyrido[4,3-d]pyrimidin-5-one (HSP990) or a pharmaceutically acceptable salt thereof.

The present invention further relates to a kit comprising a compound of formula (I), particularly S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide), or a pharmaceutically acceptable salt thereof, and at least one Hsp90 inhibitor or a pharmaceutically acceptable salt thereof, and a package insert or other labeling including directions for treating a proliferative disease.

The present invention further relates to a kit comprising a compound of formula (I), particularly (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide), or a pharmaceutically acceptable salt thereof, and a package insert or other labeling including directions for treating a proliferative disease by co-administering at least one Hsp90 inhibitor or a pharmaceutically acceptable salt thereof.

The following Examples illustrate the invention described above; they are not, however, intended to limit the scope of the invention in any way. The beneficial effects of the pharmaceutical combination of the present invention can also be determined by other test models known as such to the person skilled in the pertinent art.

### Example 1

### Effect of Compound a in HGC-27 Gastric Cancer Xenograft Model in Female Athymic Nude Mice

Experiments are performed in female Hsd:Athymic Nude-FoxN1^{nu} Nude mice approximately 8-12 weeks of age at treatment start. All animals are purchased from Harlan (South Easton, Mass.) and are housed under Optimized Hygienic conditions in filtered top microisolator cages (maximum 5 animals per cage) with free access to food and water.

HGC-27 cells, which are human gastric carcinoma cells with a PIK3CA mutation (c1624G>A, p.E542K) and PTEN null, are grown in MEM culture medium containing 1% non-essential amino acid with 10% heat-inactivated FCS, and are incubated at 37° C. in a 5% CO₂ humidified atmosphere. Cell culture reagents are purchased from Invitrogen (Carlsbad, Calif.).

HGC-27 tumors are established in vivo by injection 5×10⁶ cells in 200 μl (100 μl PBS+100 μl Matrigel) (Cat #354234, BD Bioscience, Bedford, Mass.) subcutaneous into the right flank of the animals. The efficacy experiments are started when the tumors reach an average size of approximately 230 mm³ (day 12 post cell injection).

Compound A is formulated in 0.5% Methylcellulose (MC). 80 mg of Compound A is added to 16 ml of 0.5% MC, then stirred/vortexed and sonicated in water bath sonicator for 1 h to obtain 5 mg/ml homogeneous suspension. 0.5% MC is used to dilute the 5 mg/ml solution to 2.5 mg/ml and 1.25 mg/ml for dosing. Compound A or vehicle is administered orally at a volume of 10 ml/kg. This suspension is stable for one week at room temperature.

AUY922 mesylate is formulated in D5 Water. The correction factor for the free base compound is 1.21. To prepare 50 mg/kg freebase AUY922, 60.5 mg of the AUY922 mesylate is added to 5.0 ml of D5 water, and then is sonicated in a water bath sonicator until the solution is clear. AUY922 is administered intravenous (i.v.) at a volume of 5 ml/kg, twice a week. AUY922 is prepared fresh every time.

Tumor volumes are measured with calipers and determined according to the formula: length×diameter²×π/6. Antitumor activity is expressed as T/C % which is determined according to the formula: (mean change of tumor volume of treated animals/mean change of tumor volume of control animals)×100. Regressions (%) are calculated according to the formula ((mean tumor volume at end of treatment−mean tumor volume at start of treatment)/mean tumor volume at start of treatment)×100. Body weights and tumor volumes are recorded twice a week.

Where applicable, data is presented as mean±SEM. For all tests, the level of significance is set at p<0.05. For tumor volumes, comparisons between treatment groups and vehicle control group are done using one-way ANOVA followed by Dunnett's test. Tumor volumes comparisons between treatment groups are done using Kruskal-Wallis one way ANOVA post-hoc Student Newman Kuels test or Dunn's test. In the first experiment, Compound A is orally administered daily to HGC-27 subcutaneous xenografts, tumor-bearing nude mice at a dose of 12.5 mg/kg, 25 mg/kg and 50 mg/kg. Vehicle controls consist of animals receiving daily administration of 10 ml/kg of 0.5% MC, p.o. and i.v. administration of 5 ml/kg of D5W, twice a week.

Compound A administered orally at 12.5 mg/kg, 25 mg/kg and 50 mg/kg once daily produces a T/C % of 39.4%, 35.5% and 7.1% respectively (FIG. 1). AUY922 is administered at 50 mg/kg free base dose, twice a week produced TIC (%) 60.5% (FIG. 3). Combination of Compound A at 12.5 mg/kg with AUY922 at 50 mg/kg free base results in a T/C (%) of 16.6% (FIG. 3). Combination of Compound A at 25 mg/kg with AUY922 at 50 mg/kg free base results in a 29.48% tumor regression (FIG. 5); and combination of Compound A at 50 mg/kg with AUY922 at 50 mg/kg free base results in a 85.1% tumor regression (FIG. 7). Day 23 is the last day of tumor measurement.

Compound A produces a statistically significant antitumor effect with doses of 50 mg/kg as compared to the vehicle treated group (p<0.05, ANOVA, post hoc Dunnet's). (See FIG. 1). Compound A administered orally at 12.5, 25 and 50 mg/kg once daily produces a mean change of tumor volume of 515±85 mm³, 465±111 mm³, and 93±77 mm³ (p<0.05, ANOVA and post-hoc Dunnett's) respectively as compared to vehicle (mean change of tumor volume of 1309±169 mm³)(See FIG. 1). AUY922 produces a mean change of tumor volume 792.2±159 mm³.

Compound A administered orally at 12.5, 25 and 50 mg/kg once daily in combination of AUY922 at 50 mg/kg, twice a week produces a mean change of tumor volume of 217±68 mm³ (p<0.05, compared with Vehicle and both single agents by Kruskal-Wallis ANOVA post-hoc Student Newman Kuels test), −68±36 mm³ (p<0.05, compared with Vehicle and AUY922 treated group by Kruskal-Wallis one way ANOVA post-hoc Dunn's test), and −196±21 mm³ (p<0.05, compared with Vehicle and both single agents by Kruskal-Wallis one way ANOVA post-hoc Student Newman Kuels test) respectively (See FIGS. 3,5 and 7).

Compound A is well tolerated at 12.5, 25 mg/kg and 50 mg/kg as demonstrated by the body weight change for the vehicle treated group (7.8±1.4%) and the Compound A treated group (5.3±1.4%, 2.2±1.1%, and −1.1±1.6% respectively). AUY922 treated group results in a 6.6±2.6% body weight change.

Compound A administered orally at 12.5, 25 and 50 mg/kg once daily in combination of AUY922 at 50 mg/kg, twice a week is tolerated at all doses (0.9±1.5%, −3.0±2.4%, 8.06±2.4%) (See FIGS. 4,6 and 8).

### Example 2

### Effect of Compound a in HGC-27 Gastric Cancer Xenograft Model in Female Athymic Nude Mice

The procedure described in Example 1 is followed with the following modifications:

Treatments are initiated on day 20 following tumor cell implantation of 5 million HCG-27 cells, when the average tumor volume is 316 mm³ (164-485 mm³). Animals are administered either: (a) Vehicle controls consist of animals receiving daily administration of 10 ml/kg of 0.5% MC, p.o. and i.v. administration of 5 ml/kg of D5W, twice a week, (b) 50 mg/kg AUY922, 2 q.w., i.v., (c) Compound A, either 25 mg/kg or 50 mg/kg, q.d., p.o., (d) a combination of AUY922, 50 mg/kg, 2 q.w., i.v., and Compound A, 25 mg/kg, q.d., p.o. or (e) a combination of AUY922, 50 mg/kg, 2 q.w., i.v., and Compound A, 50 mg/kg, q.d., p.o. Treatments continue for 14 days.

In this experiment, Compound A at 25 and 50 mg/kg results in significant tumor growth inhibition with 11% T/C (p<0.05 s. vehicle) and 10% T/C (p<0.05 vs. vehicle) respectively. AUY922 at 50 mg/kg results in 57% T/C, which is not significant as compared with vehicle treated group. Compound A at 25 and 50 mg/kg in combination with AUY922 at 50 mg/kg results in −11% T/T0 (p<0.05 vs. vehicle or AUY922 treated groups) and −57% T/T0 (p<0.05 vs. vehicle, AUY922 or Compound A treated groups respectively.

### Example 3

### Effect of Compound a in NCI-N87 Gastric Cancer Xenograft Model in Female Athymic Nude Mice

Experiments are performed in female Hsd:Athymic Nude-nu CPB mice approximately 10-12 weeks of age at treatment start. All animals are obtained from Harlan (Winkelmann, Germany) and are housed under Optimized Hygienic conditions in Makrolon type Ill cages (maximum 5 animals per cage) with free access to food and water.

NCI-N87 cells, which are human gastric carcinoma cells, are grown in DMEM culture medium containing 4.5 g/l glucose supplemented with 10% heat-inactivated FCS, 2 mM L-glutamine, 1 mM sodium pyruvate. The cells are incubated at 37° C. in a 5% CO2 humidified atmosphere. Cells are harvested with trypsin (0.25% w/v)-EDTA (0.53 mM), are re-suspended in culture medium (with additives) and are counted with a Casy® system. Cell culture reagents are purchased from BioConcept (Allschwil, Switzerland).

NCI-N87 tumors are established by injecting 8×10⁶ to 1×10⁷ cells (in HBSS containing 50% v/v Matrigel) subcutaneously with a 23 Gauge needle. When tumors are established and reach between 180 and 210 mm³, animals are randomized into treatment groups and the treatments are initiated.

Compound A is formulated in NMP/PEG300/Solutol HS15/water (10:30:20:40% vol/vol). The compound is fully dissolved in NMP first and water is added immediately prior to administration to animals. Compound A or vehicle is administered orally at a volume of 10 ml/kg. This suspension is stable for one week at room temperature.

AUY922 mesylate is formulated in D5 Water (5% glucose in water). All doses of AUY922 refer to the free base equivalent. AUY922 is administered iv at a volume of 10 ml/kg, twice a week.

Where applicable, data is presented as mean±SEM. For all tests, the level of significance is set at p<0.05. For tumor volumes, comparisons between treatment groups and vehicle control group are done using one-way ANOVA followed by Dunnett's test. Pairwise comparisons are done using a one way ANOVA followed by Tukey's test. The level of significance of body weight change within a group between start and end of the treatment period is determined using a paired t-test. Comparison of delta body weights between treatment and vehicle control groups is performed by one-way ANOVA followed by a post-hoc Dunnett's test. Calculations are performed using GraphPad Prism 4 for windows (GraphPad Software Inc.).

In addition, an approximation of drug interactions is made using the method described by Clarke R., “Issues in experimental design and endpoint analysis in the study of experimental cytotoxic agents in vivo in breast cancer and other models”, Breast Cancer Res. Treat., 46, 255-78 (1997). This is applied to ΔTV (Tumor volume).

Tumor volumes comparisons between treatment groups are done using Kruskal-Wallis one way ANOVA post-hoc Student Newman Kuels test or Dunn's test.

First Experiment:

Female athymic nude mice are treated orally once a day with 50 mg/kg Compound A, alone or in combination with 50 mg/kg of AUY922 administered intravenously twice a week. Vehicle controls consists of animals receiving a daily oral administration of a mixture of NMP/PEG300/Solutol HS15/water (10:30:20:40% vol/vol), in addition to an intravenous administration of 10 ml/kg of a solution of 5% glucose in water.

As a single agent, Compound A produces a statistically significant antitumor effect, with a T/C of 4.2% (p<0.05, one way ANOVA, post hoc Dunnett's) and a mean change of tumor volume (mm³±SEM) of −15.1±21.4. AUY922 (50 mg/kg) used as a single agent produces 7% tumor regressions, and when combined with Compound A produces 72.3% regressions. Both effects are significantly different from vehicle controls (p<0.05, ANOVA).

Vehicle controls produce a mean change of tumor volume (mm³±SEM) of 248.9±20.4. AUY922 (50 mg/kg) used as single agent produces a mean change of tumor volume of −15.1±21.4, and Compound A used as single agent and a mean change of tumor volume (mm³±SEM) of 1.4±18.8. The combination of AUY922 and Compound A produces a mean change of tumor volume (mm³±SEM) of −155.8±14.7. In addition, the group treated with the combination is significantly different from both Compound A and AUY922 administered as single agents (p<0.05, one way ANOVA, post hoc Tukey's).

Moreover, an analysis of possible compound interactions with the method described by Clarke R. (1997) indicates a synergistic antitumor effect with the combination of AUY922 and Compound A:

| | | Cmpd. | AUY922 | Combo | | | A/C × | | Differ- | |
|---|---|---|---|---|---|---|---|---|---|---|
| | Vehicle | A (A) | (B) | (AB) | A/C | B/C | B/C | AB/C | ence | Result |
| ΔTV | 248.9 | 1.4 | −15.1 | −155.8 | 0.006 | −0.061 | 0.000 | −0.626 | −0.63 | synergy |

For compound A, B or the combination AB (with Vehicle Control group C), antagonism is predicted when the calculation AB/C>A/C×B/C, additive effect: AB/C═NC×B/C, synergistic interactions are predicted to occur when AB/C<NC×B/C.

The body weight change during the treatment period is statistically significant within in all the groups (p<0.05, paired t-test), with exception of the group treated with Compound A single agent. The body weight change in the combination chemotherapy group is significantly different from the body weight change in the vehicle group (one way ANOVA, post hoc Dunnett's).

Second Experiment:

In the second efficacy experiment, the tumor model is set up as in the first experiment, and the same treatment groups are used, with addition of one group treated with Compound A single agent at the dose of 12.5 mg/kg, and another group treated with the same dose of Compound A combined with AUY922 (50 mg/kg, intravenously twice per week).

AUY922 as a single agent produces a statistically significant antitumor effect, with a T/C of 4.7% (p<0.05, ANOVA). As a single agent, Compound A does not produce a statistically significant antitumor effect at low (12.5 mg/kg, T/C=30.3%) dose, but the effect becomes significant at high (50 mg/kg) dose, producing 1.2% regressions (p<0.05, ANOVA). When combined with AUY922 (50 mg/kg), Compound A at low (12.5 mg/kg) and high (50 mg/kg) doses produces a statistically significant antitumor effect, with 17.5 and 59.6% regressions, respectively. When comparing the combination groups to the single agent treatments, significant differences are found between Compound A administered at 12.5 mg/kg and the combination group.

Vehicle controls produce a mean change of tumor volume (mm³±SEM) of 378.5±57.5. AUY922 (50 mg/kg) used as single agent produces a mean change of tumor volume of 17.9±11.0. Compound A used as single agent produces a mean change of tumor volume (mm³±SEM) of 114.9±43.9 (not statistically significant) at 12.5 mg/kg and −2.3±15.2 at 50 mg/kg. The combination of AUY922 and Compound A (at 12.5 mg/kg) produces a mean change of tumor volume (mm³±SEM) of −34.8±19.5. The combination of AUY922 and Compound A (at 50 mg/kg) produces a mean change of tumor volume (mm³±SEM) of −116.2±8.3. In addition, the high dose combination group (Compound A administered at 50 mg/kg) is significantly different from both single agents (p<0.05, ANOVA).

A compound interaction analysis with the method described by Clarke (see Table 3-1 for details of the formula) is conducted and shows that a synergistic interaction occurs in both combinations as follows:
The Clarke combination index for Compound A administered at 12.5 mg/kg=−0.11,
The combination index for Compound A administered at 50 mg/kg=−0.31.

The body weight change during the treatment period is statistically significant within the vehicle, the Compound A-treated (12.5 mg/kg) and the combination (with Compound A administered at 50 mg/kg) groups (p<0.05, paired t-test). The body weight change in the group treated with Compound A (50 mg/kg) and in the combination group is significantly different from the body weight change in the vehicle group (one way ANOVA, post hoc Dunnett's).

Third Experiment:

In the third efficacy experiment, the tumor model is set up as in the second experiment.

In this experiment, AUY922 is administered as a single agent produces a statistically significant antitumor effect, with a T/C of 14% (p<0.05, ANOVA). Both doses of Compound A (12.5 and 50 mg/kg, orally once a day) produces statistically significant antitumor effects, with a T/C of 37.8% and 6.4% regressions, respectively (p<0.05, ANOVA). Significant effects are also obtained in the two combination groups, with 37.4 and 63.1% regressions for Compound A administered at 12.5 and 50 mg/kg, respectively, together with 50 mg/kg AUY922.

Vehicle controls produce a mean change of tumor volume (mm³±SEM) of 195.4±22.9. AUY922 (50 mg/kg) used as single agent produces a mean change of tumor volume of 27.4±10.2. Compound A used as single agent produces a mean change of tumor volume (mm³±SEM) of 73.9±17.6 at 12.5 mg/kg and −13.3±6.7 at 50 mg/kg. The combination of AUY922 and Compound A (at 12.5 mg/kg) produces a mean change of tumor volume (mm³±SEM) of −78.4±7.4. The combination of AUY922 and Compound A (at 50 mg/kg) produces a mean change of tumor volume (mm³±SEM) of −132.0±7.9. Both combination groups are also significantly different from both respective single agents (p<0.05, ANOVA post hoc Tukey's), but the single agents did not differ from each other.

A compound interaction analysis with the method described by Clarke 1997 is conducted and shows synergistic interaction in both combinations as follows:
The Clarke combination index for Compound A administered at 12.5 mg/kg=−0.45,
The combination index for Compound A administered at 50 mg/kg=−0.67.

The body weight change during the treatment period is statistically significant within the vehicle, the Compound A-treated (12.5 mg/kg) and the combination (with Compound A adminstered at 50 mg/kg) groups (p<0.05, paired t-test). The body weight changes in both combination chemotherapy groups, as well as in the group treated with Compound A (50 mg/kg) are significantly different from the body weight changes in the vehicle group (one way ANOVA, post hoc Dunnett's).

### Example 4

### Effect of Compound a in KYSE-70 Esophageal Squamous Cell Carcinoma Xenograft Model in Female Athymic Nude Mice

Experiments are performed in female Hsd:Athymic Nude-nu CPB mice approximately 10-12 weeks of age at treatment start. KYSE-70 tumors are established by injecting 7.5×10⁶ KYSE-70 cells, which are esophageal squamous cell carcinoma cells, in 100 μl cell suspension, with a 23 gauge needle subcutaneously on the right flank of the mice. Ten (10) days after implantation, tumors reached. When tumors are established and reach about 156 mm³ (minimum 86 mm³, maximum 218 mm³) approximately 10 days after implantation, 48 animals are selected and randomized into 6 treatment groups (n=8).

Placebo control is formulated as 10 ml/kg of 250 μl NMP, 750 μl PEG300, 500 μl Solutol HS15, and 1000 μl water for delivery once daily per oral (Placebo 1) and as 10 ml/kg of 2.5 ml Glucose for delivery intravenously twice a week (Placebo 2).

Compound A is formulated in NMP/PEG300/Solutol HS15/water (10:30:20:40% vol/vol). The compound is fully dissolved in NMP first and water is added immediately prior to administration to animals. Compound A or vehicle is administered orally at a volume of 10 ml/kg. This suspension is stable for one week at room temperature.

AUY922 mesylate is formulated in D5 Water (5% glucose in water). All doses of AUY922 refer to the free base equivalent. AUY922 is administered iv at a volume of 10 ml/kg, twice a week.

Each group of mice are treated for 24 days with one of the following treatments: (a) Placebo 1 and Placebo 2 (Group 1), (b) 12.5 mg/kg Compound A per oral once daily (Group 2), (c) 50 mg/kg Compound A per oral once daily (Group 3), (d) 50 mg/kg AUY922 intravenous twice a week (Group 4), (e) a combination of 12.5 mg/kg Compound A per oral once daily and 50 mg/kg AUY922 intravenous twice a week (Group 5), or (f) a combination of 50 mg/kg Compound A per oral once daily and 50 mg/kg AUY922 intravenous twice a week (Group 6).

Antitumor activity is expressed as T/C % which is determined according to the formula: (mean change of tumor volume of treated animals/mean change of tumor volume of control animals)×100.

The following antitumor activity data is obtained with by following the above experiment protocol:

| | |
|---|---|
| Group | T/C [%] |

| No. | Day 10 | Day 14 | Day 17 | Day 21 | Day 24 |
|---|---|---|---|---|---|
| 1 | 100.0 | 100.0 | 100.0 | 100.0 | 100.0 |
| 2 | 100.0 | −7.1 | 74.0 | 40.6 | 47.5 |
| 3 | 100.0 | −49.3 | −2.7 | −40.1 | −10.9 |
| 4 | 100.0 | 145.3 | 144.4 | 131.8 | 141.1 |
| 5 | 100.0 | −17.0 | 54.6 | 18.1 | 43.1 |
| 6 | 100.0 | −53.7 | −15.9 | −39.5 | −18.7 |

### Example 5

### Effect of Compound a in A375 Melanoma Cell Carcinoma Xenograft Model in Female Athymic Nude Mice

Experiments are performed in female Harlan Hsd:Npa athymic nude mice weighing approximately 20-25 g. A375 tumors are established by injecting 4×10⁶ A375 cells, which are melanoma cells, subcutaneously on the back of the mice. Ten (10) days after implantation, tumors reached. Approximately 30 days after implantation, 32 animals are selected and randomized into 4 treatment groups (n=8).

Placebo control is formulated as 1% carboxymethylcellulose (CMC) for delivery once daily per oral (Placebo 1) and as 10 ml/kg of 2.5 ml Glucose for delivery intravenously or intraperitoneally twice a week (Placebo 2).

Compound A is formulated at a dose of 40 mg/kg Compound A by dissolving in 1% (w/v) carboxymethylcellulose (CMC) contaiing 5% (v/v) Tween-80. Compound A or vehicle is administered orally at 10 mg/ml volume once daily.

AUY922 is formulated in D5 Water (5% glucose in water). All doses of AUY922 refer to the free base equivalent. AUY922 is administered i.v. at a volume of 10 mg/ml and at a dose of 50 mg/kg, twice a week.

Each group of mice are treated for 11 days with one of the following treatments: (a) Placebo 1 and Placebo 2 (Group 1), (b) 40 mg/kg Compound A per oral once daily (Group 2), (c) 50 mg/kg AUY922 intravenous twice a week (Group 3), (e) a combination of 40 mg/kg Compound A per oral once daily and 50 mg/kg AUY922 intravenous twice a week (Group 4).

Where applicable, data is presented as mean±SEM. For all tests, the level of significance is set at p<0.05. Tumor volumes comparisons between treatment groups are done using Kruskal-Wallis One Way Analysis of Variance on Ranks or Tukey test.

Following the above experiment procedure, the mean fractional tumor growth and mean body weight change of the treated mice are shown in FIG. 9.

### Example 6

### Effect of Compound A in A375 melanoma cell carcinoma xenograft model in female athymic nude mice

Experiments are performed in female Harlan Hsd:Npa athymic nude mice weighing approximately 20-25 g. A375 tumors are established by injecting 4×10⁶ A375 cells, which are melanoma cells, subcutaneously on the back of the mice. Ten (10) days after implantation, tumors reached. Approximately 30 days after implantation, 32 animals are selected and randomized into 4 treatment groups (n=8).

Placebo control is formulated as 1% carboxymethylcellulose (CMC) for delivery once daily per oral (Placebo 1) and as 10 ml/kg of 2.5 ml Glucose for delivery intravenously or intraperitoneally twice a week (Placebo 2).

Compound A is formulated at a dose of 40 mg/kg Compound A by dissolving in 1% (w/v) carboxymethylcellulose (CMC) containing 5% (v/v) Tween-80. Compound A or vehicle is administered orally at 10 mg/ml volume once daily.

AUY922 is formulated in D5 Water (5% glucose in water). All doses of AUY922 refer to the free base equivalent. AUY922 is administered i.v. at a volume of 10 mg/ml and at a dose of 50 mg/kg, twice a week.

Each group of mice is treated for 11 days with one of the following treatments: (a) Placebo 1 and Placebo 2 (Group 1), (b) 40 mg/kg Compound A per oral once daily (Group 2), (c) 50 mg/kg AUY922 intravenous twice a week (Group 3), (d) a combination of 40 mg/kg Compound A per oral once daily and 50 mg/kg AUY922 intravenous twice a week (Group 4).

Where applicable, data is presented as mean±SEM. For all tests, the level of significance is set at p<0.05. Tumor volumes comparisons between treatment groups are done using Kruskal-Wallis One Way Analysis of Variance on Ranks or Tukey test.

Following the above experiment procedure, the mean fractional tumor growth and mean body weight change of the treated mice are shown in FIG. 9.

1. A pharmaceutical combination comprising:
(a) a compound of formula (I), [chemical structure]
wherein
A represents a heteroaryl selected from the group consisting of: [chemical structure]
R¹ represents one of the following substituents: (1) unsubstituted or substituted, preferably substituted C₁-C₇-alkyl, wherein said substituents are independently selected from one or more, preferably one to nine of the following moieties: deuterium, fluoro, or one to two of the following moieties C₃-C₅-cycloalkyl; (2) optionally substituted C₃-C₅-cycloalkyl wherein said substituents are independently selected from one or more, preferably one to four of the following moieties: deuterium, C₁-C₄-alkyl (preferably methyl), fluoro, cyano, aminocarbonyl; (3) optionally substituted phenyl wherein said substituents are independently selected from one or more, preferably one to two of the following moieties: deuterium, halo, cyano, C₁-C₇-alkyl, C₁-C₇-alkylamino, di(C₁-C₇-alkyl)amino, C₁-C₇-alkylaminocarbonyl, di(C₁-C₇-alkyl)aminocarbonyl, C₁-C₇-alkoxy; (4) optionally mono- or di-substituted amine; wherein said substituents are independently selected from the following moieties: deuterium, C₁-C₇-alkyl (which is unsubstituted or substituted by one or more substituents selected from the group of deuterium, fluoro, chloro, hydroxy), phenylsulfonyl (which is unsubstituted or substituted by one or more, preferably one, C₁-C₇-alkyl, C₁-C₇-alkoxy, di(C₁-C₇-alkyl)amino-C₁-C₇-alkoxy); (5) substituted sulfonyl; wherein said substituent is selected from the following moieties: C₁-C₇-alkyl (which is unsubstituted or substituted by one or more substituents selected from the group of deuterium, fluoro), pyrrolidino, (which is unsubstituted or substituted by one or more substituents selected from the group of deuterium, hydroxy, oxo; particularly one oxo); (6) fluoro, chloro;
R² represents hydrogen;
R³ represents (1) hydrogen, (2) fluoro, chloro, (3) optionally substituted methyl, wherein said substituents are independently selected from one or more, preferably one to three of the following moieties: deuterium, fluoro, chloro, dimethylamino;
with the exception of (S)-Pyrrolidine-1,2-dicarboxylic acid 2-amide 1-({5-[2-(tert-butyl)-pyrimidin-4-yl]-4-methyl-thiazol-2-yl}-amide),
or a pharmaceutically acceptable salt thereof; and
(b) at least one Hsp90 inhibitor or a pharmaceutically acceptable salt thereof.

2. A pharmaceutical combination according to claim 1, wherein agent (a) is selected from (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (Compound A) or a pharmaceutically acceptable salt thereof.

3. A pharmaceutical combination according to claim 1, wherein agent (b) is selected from Tanespimycin (17-allylamino-17-demethoxygeldanamycin)(also known as KOS-953 and 17-AAG); Radicicol; 6-Chloro-9-(4-methoxy-3,5-dimethylpyridin-2-ylmethyl)-9H-purin-2-amine methanesulfonate (also known as CNF2024); IPI504; SNX5422; 5-(2,4-Dihydroxy-5-isopropyl-phenyl)-4-(4-morpholin-4-ylmethyl-phenyl)-isoxazole-3-carboxylic acid ethylamide (AUY922); and (R)-2-amino-7-[4-fluoro-2-(6-methyoxy-pyridin-2-yl)-phenyl]-4-methyl-7,8-dihydro-6H-pyrido[4,3-d]pyrimidin-5-one (HSP990) or a pharmaceutically acceptable salt thereof.

4. A pharmaceutical combination according to claim 1 for simultaneous, separate or sequential use for the treatment of a proliferative disease.

5. A pharmaceutical combination according to claim 4, wherein the proliferative disease is a cancer selected from sarcoma, lung, bronchus, prostate, breast, pancreas, gastrointestine, gastric, colon, rectum, colorectal adenoma, thyroid, liver, intrahepatic bile duct, hepatocellular, adrenal gland, stomach, glioma, glioblastoma, endometrial, kidney, renal pelvis, urinary bladder, uterine corpus, uterine cervix, vagina, ovary, multiple myeloma, esophagus, a leukaemia, acute myelogenous leukemia, chronic myelogenous leukemia, lymphocytic leukemia, myeloid leukemia, brain, oral cavity, pharynx, larynx, small intestine, non-Hodgkin lymphoma, melanoma, villous colon adenoma, a neoplasia, a neoplasia of epithelial character, lymphomas, a mammary carcinoma, basal cell carcinoma, squamous cell carcinoma, actinic keratosis, a tumor of the neck or head, polycythemia vera, essential thrombocythemia, myelofibrosis with myeloid metaplasia, and Walden stroem disease.

6. A pharmaceutical composition comprising a compound of formula I according to claim 1 or a pharmaceutically acceptable salt thereof and at least one Hsp90 inhibitor or a pharmaceutically acceptable salt thereof for use in the treatment of a proliferative disease.

7. (canceled)

8. (canceled)

9. A method for treating a proliferative disease in a subject in need thereof, comprising administering to said subject a therapeutically effective amount of a compound of formula (I) according to claim 1 or a pharmaceutically acceptable salt thereof, and at least one Hsp90 inhibitor or a pharmaceutically acceptable salt thereof.

10. A method for treating a proliferative disease according to claim 9, wherein the proliferative disease is a cancer selected from sarcoma, lung, bronchus, prostate, breast, pancreas, gastrointestine, gastric, colon, rectum, colorectal adenoma, thyroid, liver, intrahepatic bile duct, hepatocellular, adrenal gland, stomach, glioma, glioblastoma, endometrial, kidney, renal pelvis, urinary bladder, uterine corpus, uterine cervix, vagina, ovary, multiple myeloma, esophagus, a leukaemia, acute myelogenous leukemia, chronic myelogenous leukemia, lymphocytic leukemia, myeloid leukemia, brain, oral cavity, pharynx, larynx, small intestine, non-Hodgkin lymphoma, melanoma, villous colon adenoma, a neoplasia, a neoplasia of epithelial character, lymphomas, a mammary carcinoma, basal cell carcinoma, squamous cell carcinoma, actinic keratosis, a tumor of the neck or head, polycythemia vera, essential thrombocythemia, myelofibrosis with myeloid metaplasia, and Walden stroem disease.

11. A method according to claim 9, wherein the compound of formula (I) is (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (Compound A) or a pharmaceutically acceptable salt thereof.

12. A method according to claim 9, wherein the Hsp90 inhibitor is selected Tanespimycin (17-allylamino-17-demethoxygeldanamycin)(also known as KOS-953 and 17-AAG); Radicicol; 6-Chloro-9-(4-methoxy-3,5-dimethylpyridin-2-ylmethyl)-9H-purin-2-amine methanesulfonate (also known as CNF2024); IPI504; SNX5422; 5-(2,4-Dihydroxy-5-isopropyl-phenyl)-4-(4-morpholin-4-ylmethyl-phenyl)-isoxazole-3-carboxylic acid ethylamide (AUY922); and (R)-2-amino-7-[4-fluoro-2-(6-methyoxy-pyridin-2-yl)-phenyl]-4-methyl-7,8-dihydro-6H-pyrido[4,3-d]pyrimidin-5-one (HSP990).

13. A method according to claim 9, wherein the compound of formula (I) and the Hsp90 inhibitor are administered together as a single pharmaceutical composition.

14. A method according to claim 9, wherein the compound of formula (I) and the Hsp90 inhibitor are administered as separate compositions or sequentially.

15. A kit comprising a compound of formula (I) according to claim 1 or a pharmaceutically acceptable salt thereof, and a package insert or label providing directions for treating a proliferative disease by co-administering at least one Hsp90 inhibitor or a pharmaceutically acceptable salt thereof.

16. A method according to claim 9, wherein the proliferative disease is a cancer of the esophagus, gastrointestine or gastric.
